(19) United States
(12) Patent Application Publication
(10) Pub. No.: US 2006/0276673 A1
(43) Pub. Date: Dec. 7, 2006

(54) Process for producing amino acid derivatives

(75) Inventors: Roland Callens, Grimbergen (BE); Marc Laroheveque, Paris (FR); Cyrille Pousset, Brussels (BE)

Correspondence Address: CONNOLLY BOVE LODGE & HUTZ, LLP, P O BOX 2207, WILMINGTON, DE, 19899, US

(73) Assignee: Solvay S.A., Brussels (BE)

(21) Appl. No.: 10/551,734

(22) PCT Filed: Apr. 2, 2004
(86) PCT No.: PCT/EP04/03687

§ 371 Date: Oct. 3, 2005

(30) Foreign Application Priority Data
Apr. 4, 2003 (FR) 03.04218

(51) Int. Cl. C07C 229/02

(52) U.S. Cl. 562/445; 562/553

(57) ABSTRACT

Process for producing amino acid derivatives, in which (a) an organic amine, the amino functionality of which is protected, or an α-amino acid, the amino functionality of which is protected, is subjected to an electrochemical reaction so as to form an amine which is activated in the α-position; (b) the activated amine is subjected to a reaction with a carbanionic reagent containing at least 3 carbon atoms and comprising an unsaturated group so as to form an unsaturated amine comprising an unsaturated group, the atom of the unsaturated group closest to the nitrogen being separated from the nitrogen by at least 2 carbon atoms; (c) the unsaturated amine is subjected to oxidation of the unsaturated group so as to form an amino acid derivative.

The present invention relates to a process for producing amino acid derivatives.

Some amino acids and their derivatives are useful in the context of the production of peptides which can be used as medicinal products.

In the search for active principles, it is desirable to have amino acids which participate in the pharmacological activity in particular of peptides and which can be used in the process for producing peptides or peptide analogues.

U.S. Pat. No. 3,891,616 describes some biologically active peptides containing 2-pyrrolidineacetic acid. The N-Boc derivative of this acid is prepared by treatment of natural L-proline with diazomethane.

This known process requires the use of a natural amino acid as starting product. The latter is subjected to conversions with a dangerous reagent under conditions which may involve a risk of racemization.

The invention is aimed at remedying the abovementioned problems.

The invention consequently relates to a process for producing amino acid derivatives, in which
(a) an organic amine, the amino functionality of which is protected, or an α-amino acid, the amino functionality of which is protected, is subjected to an electrochemical reaction so as to form an amine which is activated in the α-position;
(b) the activated amine is subjected to a reaction with a carbanionic reagent-containing at least 3 carbon atoms and comprising an unsaturated group so as to form an unsaturated amine comprising an unsaturated group, the atom of the unsaturated group closest to the nitrogen being separated from the nitrogen by at least 2 carbon atoms;
(c) the unsaturated amine is subjected to oxidation of the unsaturated group so as to form an amino acid derivative.

It has been found, surprisingly, that the process according to the invention enables efficient production of a large variety of amino acid derivatives. The initial protective group is stable under the conditions of steps (a) to (c) and is particularly useful for subsequent conversions such as racemate separation or peptide synthesis.

By way of nonlimiting examples of amino function-protecting groups which may be represented by Z, mention may in particular be made of substituted or unsubstituted groups of acyl type, such as the formyl, acetyl, trifluoroacetyl or benzoyl group, substituted or unsubstituted groups of aralkyloxycarbonyl type, such as the benzyloxycarbonyl, p-chlorobenzyl-oxycarbonyl, p-bromobenzyloxycarbonyl, p-nitrobenzyloxycarbonyl, p-methoxybenzyloxycarbonyl, benzhydryloxycarbonyl, 2-(p-biphenylyl)isopropyloxycarbonyl, 2-(3,5-dimethoxyphenyl)isopropyl-oxycarbonyl, p-phenylazobenzyloxycarbonyl, triphenylphosphonoethyl-oxycarbonyl or 9-fluorenylmethyloxycarbonyl group, substituted or unsubstituted groups of alkyloxycarbonyl type, such as the tert-butyloxycarbonyl, tert-amyloxycarbonyl, diisopropylmethyloxycarbonyl, isopropyloxycarbonyl, ethyloxycarbonyl, allyloxycarbonyl, 2-methylsulphonyl-ethyloxycarbonyl or 2,2,2-trichloroethyloxycarbonyl group, groups of cycloalkyloxycarbonyl type, such as the cyclopentyloxycarbonyl, cyclohexyloxycarbonyl, adamantyloxycarbonyl or isobornyloxycarbonyl group, and groups containing a hetero atom, such as the benzenesulphonyl, p-toluenesulphonyl (tosyl), mesitylenesulphonyl, methoxytrimethylphenylsulphonyl or o-nitrophenylsulphenyl group.

Among these groups Z, those containing a carbonyl or sulphonyl group are preferred. Acyl, aralkyloxycarbonyl and alkyloxycarbonyl groups are more particularly preferred. Among the acyl groups, an acetyl or phenylacetyl group or the like is particularly preferred. Groups similar to the phenylacetyl group are, for example, chosen from p-hydroxyphenylacetyl, p-aminophenylacetyl, furylmethyl, 2-thienylmethyl, D-α-aminobenzyl, chloroacetyl and n-propoxymethyl.

Preferably, the protective group is sterically hindering. The term “sterically hindering” is intended to denote in particular a substituent containing at least 3 carbon atoms, in particular at least 4 carbon atoms, including at least one secondary, tertiary or quaternary carbon atom. Often, the sterically hindering group contains at most 100, or even 50 carbon atoms. A protective group chosen from the alkoxycarbonyl, aryloxycarbonyl and aralkoxycarbonyl group is preferred. A tert-butyloxycarbonyl (BOC) group is most particularly preferred.

The protective group is preferably achiral or racemic.

In a first embodiment of the process according to the invention, the reaction of step (a) is carried out in the presence of the carbanionic reagent containing at least 3 carbon atoms and an unsaturated group so as to directly form an unsaturated amine comprising the unsaturated group. In this embodiment, allyltrialkylsilanes, in particular allyltrimethylsilane, are preferred as carbanionic reagent. Good results are obtained in the absence of substitution catalysts.

In a second embodiment of the process according to the invention, the activated amine is obtained by electrochemical reaction in the presence of a nucleophile so as to form an amine substituted in the α-position with the nucleophilic substituent, as activated amine, and step (b) is preferably carried out in the presence of a substitution catalyst. The nucleophile is often chosen from an alcohol and a carboxylic acid. It is preferably chosen from methanol and acetic acid. Methanol is more particularly preferred.

In this embodiment, a Lewis acid is often used as substitution catalyst. The substitution catalyst is preferably a titanium or boron compound. Titanium tetrachloride and boron-trifluoride etherate are particularly preferred.

In the process according to the invention, step (a) can be carried out in a compartmentalized or non-compartmentalized cell.

The electrodes used in step (a) should be resistant with respect to the conditions of the electrochemical reaction. Suitable materials are in particular chosen from metals, metal oxides and graphite. Particularly suitable metals are chosen from steel, iron and titanium, and in particular from the metals of the group of platinum and their oxides, or electrodes coated with the latter materials. Platinum or rhodium is preferred. An electrode comprising platinum is particularly suitable.

The distance between the electrodes is generally at least 0.2 mm. This distance is often at least 0.5 mm. It is preferably at least 1 mm. The distance between the electrodes is generally at most 20 mm. This distance is often at most 10 mm. It is preferably at most 5 mm.

In the process according to the invention, step (a) is generally carried out at a current density greater than or equal to 0.1 A/dm². The current density is often greater than or equal to 1 A/dm². It is preferably greater than or equal to 3 A/dm². In the process according to the invention, step (a) is generally carried out at a current density less than or equal to 50 A/dm². The current density is often less than or equal to 30 A/dm². It is preferably less than or equal to 20 A/dm².

In the process according to the invention, step (a) is generally carried out at a temperature greater than or equal to −50° C. The temperature is often greater than or equal to −20° C. It is preferably greater than or equal to 0° C. In the process according to the invention, step (a) is generally carried out at a temperature less than or equal to 100° C. The temperature is often less than or equal to 80° C. It is preferably less than or equal to 60° C.

In the process according to the invention, an allyl carbanionic reagent is often used in step (b).

In a first variant of the process according to the invention, the unsaturated amine comprises a carbonyl group as unsaturated group. Such unsaturated amines can be obtained, for example, when a silyl enol ether, in particular a trialkylsilyl enol ether, is used as carbanionic reagent. Trialkylsilyl enol ethers of formula
H2C═C(OSi(alkyl)₃)-R (I)
in which R denotes an alkyl, preferably sterically hindering, group or an aryl group are preferred.

In a second variant of the process according to the invention, the unsaturated amine comprises an olefin double bond as unsaturated group.

In this variant, an allyltrialkylsilane is preferably used as carbanionic reagent. Allyltrimethylsilane is more particularly preferred.

In the process according to the invention, the oxidation can, for example, be oxidation with periodate, preferably catalyzed by a metal such as ruthenium, or ozonolysis or else, when the unsaturated amine comprises a carbonyl group, Baeyer-Villiger oxidation, for example with a peracid such as peracetic acid or trifluoroperacetic acid. Oxidative cleavage by ozonolysis is particularly preferred.

The invention also relates to a process for producing enantiopure amino acid derivatives, comprising the steps:
(a) a racemic amino acid derivative is produced according to the process of the invention;
(b) the enantiomers of the racemic amino acid derivative are separated.

In this process, the separation of the enantiomers can be carried out, for example, by enzymatic reaction. Suitable enzymes are chosen, for example, from oxydoreductases, transferases, hydrolases, lyases, isomerases and ligases. Enzymatic reaction with a penicillinase or a lipase is preferred.

More particularly preferably, the process according to the invention is applied to the production of a β-amino acid derivative, in particular an enantiopure β-amino acid derivative. The process according to the invention can also be used to obtain other amino acids exhibiting a greater distance between the amino group and the carboxyl group, such as γ- δ- or ε-amino acids. The process according to the invention is suitable for obtaining cyclic or acyclic amino acids, it being possible for the amino group to be present within a heterocycle. The process is particularly suitable for the manufacture of acyclic aminoacides, in particularly when a penicillinase is used to separate enantiomers.

Specific examples of amino acids which can be obtained-according to the process according to the invention are chosen, for example, from β-homovaline, β-homophenylalanine, ε-trifluoroacetyl-β-homolysine, β-homolysine, β-homoaspartic acid, β-homoproline, pyrrolidine-2-acetic acid and 2-piperidineacetic acid.

The examples below are intended to illustrate the invention without, however, limiting it.

### EXAMPLE 1

### 1.1 Synthesis of N-(1-methoxy-2-methylprop-1-yl)-2-phenylacetamide

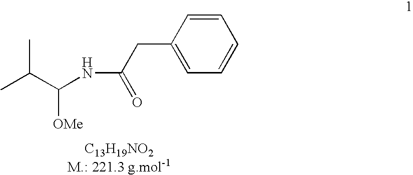

1.81 ml of triethylamine (13 mmol, 0.15 equiv.) were added to a solution of 20 g of N-phenylacetylated valine (85 mmol, 1 equiv.) in 80 ml of methanol. The mixture was cooled to around 5° C. by circulating ice-cold water around the electrode. A current of 2.8 A for a voltage of ±10 V was then applied for a period of time corresponding to two faradays, followed by a current of 1.4 A for the period of time corresponding to 0.2 faraday. The reaction was monitored by HPLC. After concentration on a rotary evaporator, the residue was diluted in 150 ml of dichloromethane and the solution was washed with 150 ml of a 5% sodium hydrogen carbonate solution. The aqueous phase was extracted twice with 100 ml of dichloromethane. The organic phases were pooled, washed with 150 ml of brine, and dried with magnesium sulphate, filtered and evaporated. Recrystallization of the brown-coloured crude product from an equimolar ethyl acetate/isooctane mixture gave 17.5 g of a white solid corresponding to the expected product (chemical yield: 93%; electrical yield: 91%).

M.p=82° C.

¹³C NMR: δ (CDCl₃) 171.4 (s, CO), 134.6 (s, C_{aromatic}), 129.2 (s, CH_{aromatic}), 129.0 (s, CH_{aromatic}), 127.4 (s, C_{para-aromatic}), 85.1 (s, CHOMe), 55.8 (s, OCH₃), 43.9 (s, NHCOCH₂Ph); 32.8 (s, (CH₃)₂CH), 17.5 & 16.9 (2s, (CH₃)₂CH).

¹H NMR: δ (DMSO) 8.19 (d, ³J_{H-H}=9.4 Hz 1H, NH), 7.3-7.1 (m, 5H_{aromatic}), 4.62 (dd, ³J_{H-H}=9.4 Hz, ³J_{H-H}=6.8 Hz, 1H, CHOMe), 3.49 (s, 2H, NHCOCH₂Ph), 3.11 (s, 3H, OCH₃), 1.74 (dq, ³J_{H-H}=6.8 Hz, ³J_{H-H}=6.7 Hz, 1H, (CH₃)₂CH), 0.85 & 0.80 (2d, ³J_{H-H}=6.7 Hz, ³J_{H-H}=6.8 Hz, 6H, (CH₃)₂(CH).

Mass spectrometry: M/Z (ESI): 465 ((2M+Na)⁺), 379 ((2M-2MeOH+H)³⁰), 244 ((M+Na)⁺). M/Z (EI): 206 (2%) ((M−Me)⁺), 189 (3%) ((M-HOMe)⁺), 178 (30%) ((M-C₃H₇)⁺)⁺), 136 (2%), 91 (37%) ((C₇H₇)⁺), 87 (63%) ((M-NHCOCH₂Ph)⁺), 72 (20%), 65 (15%), 60 (100%), 55 (19%).

I.R.: (KBr) 3276 (νNH), 1651 (νCO_{amide}).

Elemental analysis:

Calculated: C 70.56%; H 8.65%; N 6.33%

Measured: C 70.42%; H 8.67%; N 6.32%.

### 1.2. Synthesis of 2-methyl-3-phenylacetamidohex-5-ene

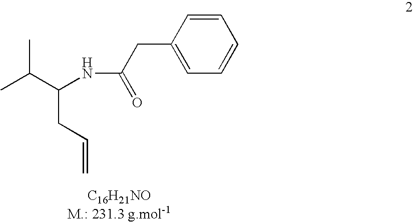

3.7 ml of titanium tetrachloride (0.034 mol, 1.4 equiv.) diluted in 10 ml of dichloromethane were added to a solution of 5.3 g of aminal 1 (0.024 mol, 1 equiv.) and of 10.3 ml of allyltrimethylsilane (0.065 mol, 2.7 equiv.) in 50 ml of dichloromethane cooled to −40° C. When the addition was complete, the solution was stirred at −40° C. for 15 min, then the mixture was left to return to ambient temperature, and the stirring was continued for 15 h. The reaction mixture was then diluted in 20 ml of dichloromethane and hydrolyzed with 6 g of calcium carbonate dissolved in 15 ml of water. The aqueous phase was extracted twice with 30 ml of dichloromethane. The organic phases were pooled, dried with magnesium sulphate, filtered and evaporated. The residue obtained was chromatographed on a silica column with, as eluent: 7/3 cyclohexane/ethyl acetate. 5.13 g of a white solid were obtained, corresponding to the expected product (yield=93%).

M.p=47° C.

¹³C NMR: δ (CDCl₃) 170.4 (s, C═O), 135.0 (s, C_{aromatic}) 134.4 (s, CH═CH₂), 129.3 (s, CH_{aromatic}), 128.9 (s, CH_{aromatic}), 127.2 (s, C_{para-aromatic}), 117,2 (s, CH═CH₂), 53.4 (s, CHNH, 43.9 (s, NHCOCH₂Ph), 36.3 (s, CH₂CH═CH₂), 31.1 (s, (CH₃)₂CH), 19.1 & 17.7 (2s, (CH₃)₂CH).

¹H NMR: δ (CDCl₃) 7.38-7.21 (m, 5H_{aromatic}), 5.64 (m, 1H, CH═CH2), 5.32 (d, ³J_{H-H}=8.6 Hz, 1H, NH), 4.92 (m, 2H CH═CH), 3.81 (m, 1H, CHNH), 3.55 (s, 2H, NHCOCHPh), 2.19 & 2.01 (2m, 2H, CH₂CH═CH₂), 1.64 (dt. ³J_{H-H}=6.7 Hz, ³J_{H-H}=13.4 Hz, 1H (CH₃)₂CH), 0.82 & 0.74 (2d, ³J_{H-H}=6.8 Hz, ³J_{H-H}=6.9 Hz, 6H, (CH₃)₂CH).

Mass spectrometry: M/Z: (ICP/NH₃) 232 ((M+H)⁺), 249 ((M+NH₄)⁺. M/Z (EI): 279 (7%) ((M)⁺), 238 (7%) ((M-C₃H₅)⁺), 188 (22%) ((M-CH₂Ph)⁺), 120 (25%), 91 (57%) ((C₇H₇)⁺),70 (100%), 65 (15%).

I.R.: (KBr) 3292 (νNH), 1643 (νCO νC═C).

Elemental analysis:

Calculated: C 77.88%; H 9.15%; N 6.05%

Measured: C 77.86%; H 9.18%; N 6.06%.

### 1.3 Synthesis of 4-methyl-3-phenylacetamidopentanoic acid

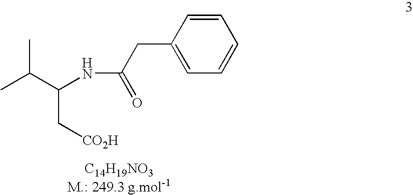

A stream of ozone was passed, via an ozonizer, through a solution of 2 g of amide 2 (8.7 mmol, 1 equiv.) in 10 ml of a dichloromethane/methanol (3/2) mixture cooled to around −70° C. with a dry ice/acetone bath. The reaction was monitored by TLC. After three hours at −70° C., the solution was degassed and then evaporated under cold conditions in a rotary evaporator so as to give a yellow oil. 5.6 ml of formic acid and 2.8 ml of hydrogen peroxide were then added and the mixture was brought to reflux for thirty minutes. After stirring overnight at ambient temperature, the solvent was first evaporated off at 60° C. under vacuum. The residue was then recrystallized from a mixture of ethyl acetate/isooctane so as to give 2.1 g of crystals corresponding to the expected product (yield: 95%).

M.p=131° C.

¹³C NMR: δ (CDCl₃) 175.8 (s, COOH), 171.9 (s, NHCOCH₂Ph), 134.4 (s, C_{aromatic}), 129.3 (s, CH_{aromatic}), 128.9 (s, CH_{aromatic}), 127.3 (s, C_{para-aromatic}), 51.7 (s, CHNH), 43.3 (s, NHCOCH₂Ph), 36.3 (s, CH₂COOH), 31.2 (s, CH₃)₂CH), 19.1 & 18.4 (2s, (CH₃)₂CH).

¹H NMR: δ (CDCl₃) 7.35-7.22 (m, 5H_{aromatic}), 6.18 (d, ³J_{H-H}=9.3 Hz, 1H, NH), 4.03 (m, 1H, CHNH), 3.61 (s, 2H, NHCOCH₂Ph), 2.53 & 2.44 (2dd. ³J_{H-H}=5.1 Hz, ³J_{H-H}=6.2 Hz, ³J_{H-H}=15.8 Hz, 2H, CH₂COOH), 1.74 (dt, ³J_{H-H}=6.9 Hz, ³J_{H-H}=8.2 Hz, 1H, (CH₃)₂CH), 0.86 & 0.79 (2d, ³J_{H-H}=6.9 Hz, ³J_{H-H}=6.8 Hz, 6H, (Ch₃)₂CH).

Mass spectrometry: M/Z (ICP/NH₃): 250 ((M+H)⁺), 267 ((M+NH₄)⁺). M/Z (EI): 249 (5%) (M)⁺), 206 (14%) ((M-C₃H₅)⁺), 190 (17%), 158 (9%) ((M-CH₂Ph)⁺), 140 (5), 136 (18%), 116 (6%), 97 (10%), 91 (100%) ((C₇H₇)⁺), 88 (87%), 73 (23%), 69 (35%), 65 (31%), 55 (15%), 41 (19%).

I.R.: (nujol) 3500-2500 (νOH, 3200 (νNH), 1700 (νCO_{acid}), 1632 (νCO_{amide}).

Elemental analysis:

Calculated: C 67.45%; H 7.68%; N 5.62%

Measured: C 67.28%; H 7.66%; N 5.62%.

### 1.4 Cleavage of the racemate (3R)-3-amino-4-methylpentanoic acid

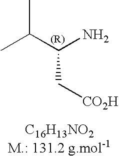

1 ml of suspension of penicillin acylase ChiroCLEC-EC® was added to a solution of 500 mg of N-phenylacetylated β-homovaline 3 (2 mmol) in 3 ml of isopropanol, 7 ml of 10⁻² M buffer solution, pH 8, and 2 ml of water. The reaction medium was stirred at 28° C. and the pH was maintained at pH 8 by means of an autotitrator, by adding a 0.1 N sodium hydroxide solution. After stirring for 24 hours, the reaction medium was centrifuged, making it possible to separate the solution from the enzyme. The solution was concentrated and the aqueous phase was then acidified to pH 2 and extracted with 3 times 10 ml of ethyl acetate. The organic phases were pooled and dried over magnesium sulphate. After evaporation, the residue was purified by flash chromatography (cyclohexane/ethyl acetate/formic acid 1/1/0.01) in order to separate the phenylacetic acid from the substrate (yield=46%). The aqueous phase was lyophilized and the residue was chromatographed on Dowex 50H⁺ resin to give the neutral amino acid (yield=45%).

M.p=206° C. [α]_{D}²⁰ =+47 (c=1; H₂O); litt.¹:

[α]_{D}²⁰ =40.3 (c=1.02; H₂O).

¹³C NMR: δ (D₂O): 179.6 (s, COOH), 55.9 (s, CHNH₂), 37.1 (s, CH₂CO₂H, 31.1 (s, (CH₃)CH), 18.5 & 18.3 (2s, (CH₃)₂CH).

¹H NMR: δ (D₂O): 3.12 (ddd, ³J_{H-H}=4.3 Hz, ³J_{H-H}=6 Hz, ³J_{H-H}=9.3 Hz, 1H, CHNH₂), 2.37 (dd, ³J_{H-H}=4.3 Hz, ³J_{H-H}=16.8 Hz, 1H of CH₂CO₂H), 2.19 (dd, ³J_{H-H}=9.3 Hz, ³J_{H-H}=16.8 Hz, 1H of CH₂CO₂H), 1.73 (dq, ³J_{H-H}=6.8 Hz, ³J_{H-H}=6.4 Hz, 1H, (CH₃)CH), 0.79 & 0.78 (2d, ³J_{H-H}=6.8 Hz, 6H, (CH₃)₂CH).

I.R: (KBr) 3300-2000 (νOH_{acid}), 3000-2000 (νNH), 1625 (νNH₂),1556 (νCOO⁻_{carboxylate}), 1399 (νCOO⁻_{carboxylate}).

Elemental analysis:

Calculated: C 54.94%; H 9.99%; N 10.68%

Measured: C 54.79%; H 10.02%; N 10.78%.

### (3S)-4-methyl-3-phenylacetamidopentanoic acid

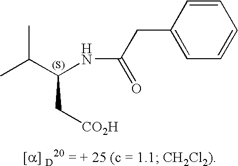

The enantiomeric excesses of the 4-methyl-3-phenylacetamidopentanoic acid were measured on the compound amidated with (R)-naphthylethylamine by HPLC. The enantiomeric excess was greater than 99%.

Elution conditions: Macherey-Nagel Nucleosil 50-5 column; mobile phase: hexane/EtOAc; 2/3. flow rate: 2 ml/min.; detection λ=265 nm; t_{R}=7.3 min for (S,R), 15.6 min for (R,R).

### EXAMPLE 2

### 2.1 Synthesis of 2-methoxy-1-phenylacetylpyrrolidine

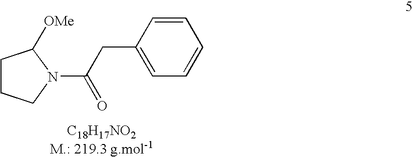

0.5 g of tetrabutylammonium tetrafluoroborate was added to a solution of 22 g of N-phenylacetylated pyrrolidine (116 mmol, 1 equiv.) in 60 ml of methanol, so as to attain a current of 2.8 A for a voltage of ±10 V. The current was maintained for the amount of time required to provide a total of 3 faradays. After concentration in a rotary evaporator (bath temperature less than 35° C.), the residue was diluted in 100 ml of dichloromethane and the solution was washed with 130 ml of water. The aqueous phase was extracted with dichloromethane. The organic phases were pooled, washed with 150 ml of brine and dried with magnesium sulphate, filtered and evaporated to give 24.3 g of a black oil. The residue was chromatographed on a silica column; eluent: 3/2 cyclohexane/ethyl acetate. 18.3 g of expected product were isolated (chemical yield: 72%; electrical yield: 87%).

¹³C NMR: δ (CDCl₃) mixture of two conformers: 1/1: 171.2 & 170.7 (2s, C═O), 135.0 & 134.4 (2s, 2C_{aromatic}), 129.1 & 129.0 (2s, 2CH_{aromatic}), 128.5 & 128.4 (2s, 2CH_{aromatic}) 126.7 & 126.6 (2s, 2C_{para-aromatic}), 88.6 & 87.2 (2s, CHOMe), 56.5 & 53.8 (2s, OCH₃), 46.2 & 45.7 (2s, NCH₂), 42.0 & 41.1 (2s, NCOCH₂Ph), 31.3 & 30.7 (2s, CH₂CH), 22.9 & 20.9 (2s, CH₂CH₂CH).

¹H NMR: δ (CDCl₃) mixture of two conformers: 1/1: 7.32-7.25 (m, 5H_{aromatic}), 5.47 & 4.99 (2d, ³J_{H-H}=4.7 Hz, 1H of a conformer, ³J_{H-H}=4.8 Hz 1H of a conformer, 1H, CHOMe), 3.78 & 3.76 (2d, ²J_{H-H}=15 Hz, ³J_{H-H}=13.8 Hz, 2H of a conformer, NCOCH₂Ph), 3.66 (s, 2H of a conformer, NCOCCH₂Ph), 3.67-3.37 (m, 2H, NCH₂), 3.39 & 3.31 (2s, 3H, OCH₃), 2.17-1.70 (3m, 4H, CH₂CH₂CH).

Mass spectrometry: M/Z (ESI): 461 ((2M+Na)⁺), 439 ((2M+H)⁺), 407 ((2M−MeOH+H)⁺), 375 ((2M−2MeOH+H)⁺), 242 ((M+Na)⁺), 220 ((M+H)⁺), 188 ((M−MeOH+H)⁺).

I.R: (pure) 1655 (νCO).

Elemental analysis:

Calculated: C 71.21%; H 7.81%; N 6.39%

Measured: C 67.70%; H 8.00%; N 5.60%.

### 2.2 Synthesis of 2-allyl-1-phenylacetylpyrrolidine

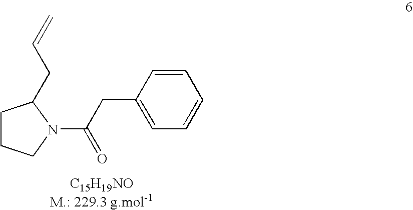

The procedure used for preparing the acetamide 2 was reproduced with 2.4 g of 2-methoxy-1-phenylacetylpyrrolidine 5 (11 mmol, 1 equiv.) and 4.5 ml of allyltrimethylsilane (28 mmol, 2.6 equiv.) in 25 ml of dichloromethane. After the addition of 2 ml of titanium tetrachloride (16 mmol, 1.4 equiv.) and stirring for 12 h at ambient temperature, the reaction was stopped and the reaction medium was treated as indicated above. After evaporation of the organic phases, 2.5 g of expected product were isolated (yield: 99%). ¹³C NMR: δ (CDCl₃) mixture of two conformers: 4/1: 169.3.(s, C═O), 135.2 & 134.9 (2s, CH═CH₂), 134.0 (2S, C_{aromatic}), 128.8 (s, CH_{aromatic}), 128.4 (s, CH_{aromatic}), 126.5 (s, Cₚₐᵣₐ₋ₘₒᵤₙₜ), 118.0 & 117.1 (2s, CH═CH₂)) 57.4 & 56.7 (2s,CHNH), 47.2 & 45.6 (2s, NCOCH₂Ph or CH₂CH═CH₂ or CH₂N), 42.5 & 41.4 (2s, NCOCH₂Ph or CH₂CH═CH₂ or CH₂H), 39.2 & 37.1 (2s, NCOCH₂Ph or CH₂CH═CH₂ or CH₂N), 29.8 & 28.4 (2s, CH₂CH), 23.8 & 21.6 (2s, CH₂CH₂N).

¹H NMR: δ (CDCl₃) mixture of two conformers: 4/1: 7.33-7.23 (m, 5H_{aromatic}), 5.81-5.69 (m, 1H, CH═CH₂), 4.21-4.15 & 3.97-3.93 (2m, 1H, CHN), 3.74-3.62 (4d, ²J_{H-H}=14.9 Hz, ²J_{H-H}=10.6 Hz, ²J_{H-H}=10.7 Hz, ²H_{H-H}=9.3 Hz, 2H, NCOCH₂Ph),

Mass spectrometry: M/Z: (ICP/NH₃) 230 ((M+H)⁺), 247 ((M+NH₄)⁺).

I.R.: (pure) 1639 (νCO, νC═C).

Elemental analysis:

Calculated: C 78.561%; H 8.35%; N 6.11%

Measured: C 78.39%; H 8.54%; N 6.11%.

### 2.3 Synthesis of carboxymethyl-1-phenylacetylpyrrolidine

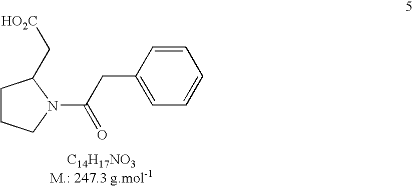

The procedure described for preparing the NV-phenylacetylated β-homovaline 3 was reproduced with 1.15 g of N-phenylacetylated 2-allylpyrrolidine 5 (5 mmol, 1 equiv.). After ozone had been passed through for two hours at −70° C., the reaction was stopped and the reaction medium was treated as indicated. After evaporation, 1.2 g of the expected product were obtained (yield: 97%).

¹³C NMR: δ (CDCl₃) mixture of two conformers: 9/1: 176.6 & 175.6 (2s, COOH), 171.1 (s, C═O), 134.3 & 133.9 (2s, C_{aromatic}), 130.0 (s, CH_{aromatic}), 128.7 (s, CH_{aromatic}), 126.9 (s, C_{para-aromatic}), 54.9 & 54.3 (2s, CHN), 47.4 & 45.8 (2s,NCOCH₂Ph or CH₂CO₂H or CH₂N), 43.1 & 42.0 (2s, NCOCH₂Ph or CH₂CO₂H or CH₂N),39.2 & 37.7 (2s, NHCOCH₂Ph or CH₂CO₂H or CH₂N), 30.2 & 28.7 (2s, CH₂CH), 23.7 & 21.3 (2s, CH₂CH₂N).

¹H NMR: δ (CDCl₃) mixture of two conformers: 9/1: 10.25 (broad, 1H OH), 7.38-7.23 (m, 5H_{aromatic}), 4.46 (1H m, CHCH₂CO₂H), 3.70 (s, 2H NCOCH₂Ph), 3.45 (m, 2H, CH₂N), 3.00 (dd, ³J_{H-H}=4.1 Hz, ²J_{H-H}=15.6 Hz, 1H of CH₂CO₂H), 2.38 (dd, ³J_{H-H}=8.8 Hz, ²J_{H-H}=15.6 Hz, 1H of CH₂CO₂H), 2.17-1.77 (m, 4H, CH₂CH₂CH),

Mass spectrometry: M/Z: (ICP/NH₃) 248 ((M+H)⁺), 265 ((M+NH₄)⁺).

1. Process for producing amino acid derivatives, in which
(a) an organic amine, the amino functionality of which is protected, or an α-amino acid, the amino functionality of which is protected, is subjected to an electrochemical reaction so as to form an amine which is activated in the α-position;
(b) the activated amine is subjected to a reaction with a carbanionic reagent containing at least 3 carbon atoms and comprising an unsaturated group so as to form an unsaturated amine comprising an unsaturated group, the atom of the unsaturated group closest to the nitrogen being separated from the nitrogen by at least 2 carbon atoms;
(c) the unsaturated amine is subjected to oxidation of the unsaturated group so as to form an amino acid derivative.

2. Process according to claim 1, in which the amino functionality is protected by a protective group comprising a carbonyl group.

3. Process according to claim 2, in which the protective group is an acyl group.

4. Process according to claim 2, in which the protective group is an alkoxycarbonyl group, an aryloxycarbonyl group or an aralkoxycarbonyl group.

5. Process according to claim 1, in which the activated amine is obtained by electrochemical reaction in the presence of a nucleophile so as to form an amine substituted in the a-position with a nucleophilic substituent, as activated amine, and step (b) is carried out in the presence of a substitution catalyst.

6. Process according to claim 5, in which the nucleophile is chosen from an alcohol and a carboxylic acid.

7. Process according to, in which an allyl carbanionic reagent is an allyltrialkylsilane, is used in step (b).

8. Process according to claim 1, in which the unsaturated amine comprises a carbonyl group as unsaturated group.

9. Process according to claim 1, in which the unsaturated amine comprises an olefin double bond as unsaturated group.

10. Process according to claim 9, in which the oxidation is oxidative cleavage by ozonolysis.

11. Process for producing amino acid derivatives, comprising steps:
(a) a racemic amino acid derivative is produced according to the process of claim 1;
(b) the enantiomers of the racemic amino acid derivative are separated.

12. Process according to claim 11, in which the separation of the enantiomers is carried out by enzymatic reaction.

13. Process according to claim 1, in which the product obtained is β-amino acid derivative.

14. Process according to claim 2, in which the protective group is acetyl or phenylacetyl group.

15. Process according to claim 2, in which the protective group is a tert-butyloxycarbonyl (BOC) group.

16. Process according to claim 5, wherein said catalyst is a titanium compound.

17. Process according to claim 5, in which the nucleophile is chosen from methanol and acetic acid.

18. Process according to claim 12, wherein the enzymatic reaction is with a penicillinase or a lipase.
